(19) United States
(12) Patent Application Publication
(10) Pub. No.: US 2009/0260630 A1
(43) Pub. Date: Oct. 22, 2009

(54) Sleep inducing apparatus for CPAP machine

(76) Inventors: Edwin Saldivar, Bronx, NY (US); Karl F. Milde, JR., Mahopac, NY (US)

Correspondence Address: MILDE & HOFFBERG, LLP, 10 BANK STREET, SUITE 460, WHITE PLAINS, NY, 10606, US

(21) Appl. No.: 12/315,325

(22) Filed: Dec. 2, 2008

Related U.S. Application Data
(63) Continuation-in-part of application No. 12/152,586, filed on May 15, 2008.
(60) Provisional application No. 61/124,932, filed on Apr. 18, 2008.

(51) Int. Cl. A61F 5/56; A61M 16/00

(52) U.S. Cl. 128/204.23; 128/207.18; 128/848

(57) ABSTRACT

A continuous positive airway pressure (“CPAP”) machine has an electrically driven air pump, an air hose coupled to the air pump, and a air mask coupled to the air hose and adapted to be applied to a head of a patient to supply a stream of air to breathing passages of said patient while the patient is sleeping. A device, attached to said machine, is provided for inducing sleep in said patient.

### CROSS-REFERENCE TO RELATED APPLICATION

This application is a continuation-in-part of application Ser. No. 12/152,586, filed May 15, 2008, entitled “SLEEP INDUCING APPARATUS FOR A CPAP MACHINE”. This application also claims priority from the U.S. Provisional Application No. 60/998,519 filed Oct. 11, 2007 and U.S. Provisional Application No. 61/124,932, filed Apr. 18, 2008.

### BACKGROUND OF THE INVENTION

The present invention relates to a continuous positive airway pressure (CPAP) machine of the type which is used by patients for the treatment for sleep apnea. The CPAP machine delivers a stream of air to the breathing passages of a patient via a hose to a nasal “pillow”, nose mask or full face mask. As used herein, any type of mask which is used to supply the stream of air to breathing passages of a patient will be denominated an “air mask”.

The CPAP machine blows air at a prescribed pressure, sometimes called the “titrated pressure”, which is sufficient to prevent most, if not all, apneas of a patient.

Optional features on a CPAP machine may include:

1. A device for automatically titrating or tuning the amount of pressure delivered to the patient to the minimum required to maintain an unobstructed airway on a breath-by-breath basis;

2. A bi-level control device which provides two levels of pressure: one for inhalation and a lower pressure during exhalation, to reduce the effort required for breathing;

3. A device that controls the number of breaths permitted of the patient;

4. A humidifier for adding moisture to the air supplied to the patient;

5. A heater for heating the air delivered to the patient; and

6. An electronic storage device for logging compliance of the patient and other operational details, allowing the sleep physician to download and analyze data to access treatment effectiveness.

All of these enhancements are well known and are intended to be included within the term “CPAP machine” as this term is used hereinafter.

While CPAP machines are very effective in treating sleep apnea, a condition which occurs when the upper airway of a patient becomes narrow as the muscles relax naturally during sleep, they have little or no effect in assisting a patient in falling asleep. Indeed, the very presence of the CPAP machine is often a hindrance or barrier to initiating sleep by a patient, due to the stress involved in applying the air mask and engaging the machine, and due to the slight discomfort of the air mask itself.

Further, a CPAP machine has little or no effect in inducing sleep for a person with insomnia. This serious condition has been documented by many sleep therapists and has been reported, for example, in the book Insomniac by Gayle Greene (2008).

### SUMMARY OF THE INVENTION

It is a principal object of the present invention to provide a CPAP machine which not only assists patients in sleeping, but also induces the sleep of the patient when the CPAP machine is utilized.

This object, as well as other objects which will become apparent from the discussion that follows, are achieved, in accordance with the present invention, by providing a device, which is built into and operative in conjunction with the CPAP machine, for inducing sleep in the patient when the machine is used; e.g., when the machine is turned on and/or when the air mask is applied to the patient's head.

The present invention thus combines a sleep inducing feature with a CPAP machine. This feature may include one or more of the following:

(1) An audio system for providing sleep-inducing sounds (such as white noise, ocean beach sounds, train sounds, water sounds, wind sounds, soft music, spoken words and/or birds songs).

(2) A vibratory massage system for massaging a body part (such as the scalp, neck, forehead, shoulders and/or back).

(3) An electrical contact system for providing electrical pulses or waves to a body part (such as the scalp, neck and/or forehead).

(4) A device for providing a sleep-inducing aroma or gas to the patient (such as a gas containing a pleasing or otherwise sleep-inducing aroma, oxygen, nitrous oxide and/or a sleeping gas such as halothane vapor (Fluothane), methyl propyl ether(Neothyl) and methxyflurane(Penthrane)).

(5) An electrical contact system for receiving electrical signals from the scalp, representative of brain activity, for controlling devices (1)-(4) above.

The sleep inducing feature (audio, vibratory, electrical, gaseous or other) is preferably attached to the CPAP machine and connected to an instrument (nose mask, loudspeaker, vibrating element and/or electrical contacts) on the head strap.

For a full understanding of the present invention, reference should now be made to the following detailed description of the preferred embodiments of the invention as illustrated in the accompanying drawings.

### BRIEF DESCRIPTION OF THE DRAWINGS

FIG. 1 is a block diagram showing a CPAP machine incorporating sleep inducing apparatus according to the present invention.

FIG. 2 shows a person wearing an air mask of a CPAP machine which incorporates components of the sleep inducing apparatus according to the invention.

### DESCRIPTION OF THE PREFERRED EMBODIMENTS

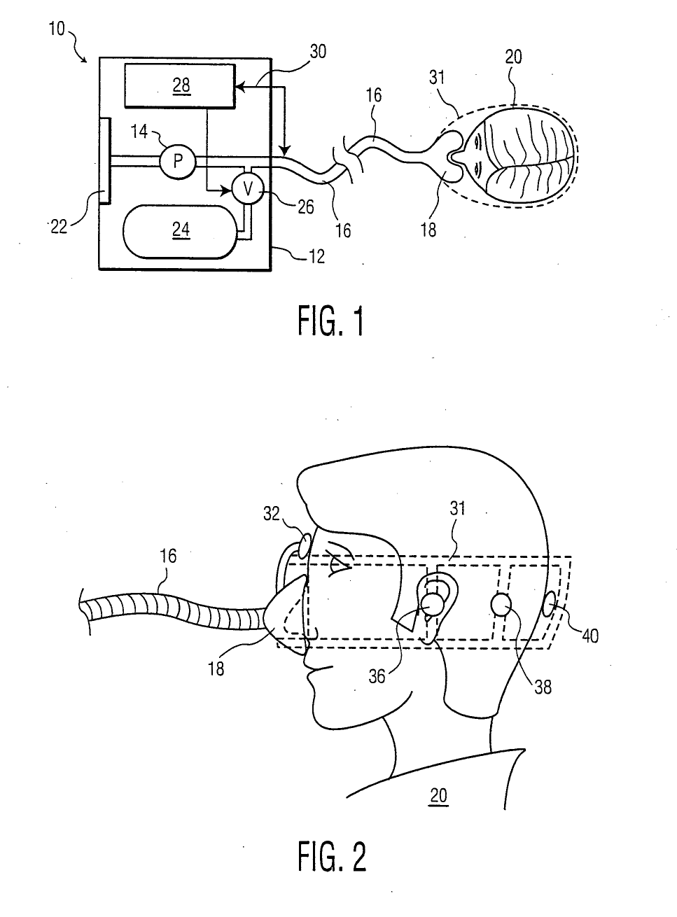

Preferred embodiments of the present invention will now be described with reference to FIGS. 1 and 2 of the drawings. Identical elements in the various figures are designated with the same reference numerals.

FIG. 1 shows a CPAP machine 10 having a housing 12 incorporating an air pump 14 that delivers a stream of air through a hose 16 to an air mask 18 worn by a patient 20. The air pump 14 receives ambient air through an air filter 22 incorporated in the housing 12. In this embodiment, the air mask comprises a nose mask 18 that covers the patient's nose and is supported by a strap 31 (shown in dashed lines) which surrounds the patient's head.

As used herein, the term “nose mask” is intended to mean and include any type of mask which is used to supply air, and/or other gases, preferably under continuous pressure, to the breathing passages of a patient.

According to the invention, the CPAP machine 10 includes a device for inducing sleep in the patient. This device may be incorporated, wholly or in part, in the housing 12 and/or attached and supported by the head strap 31 which retains the nose mask 18. Similarly, the device may be incorporated wholly or in part in the head strap and/or nose mask.

In particular, FIG. 1 shows a source of vapors; for example, a tank 24 that holds a sleep-inducing gas under pressure and/or a liquid perfume. A valve 26, coupled to an outlet of the tank, controls the flow of vapors supplied to the hose 16. The valve may be mechanically or electrically controlled by the patient or controlled electrically by an electronic device 28, which is also incorporated into the housing 12. The electronic device 28 may receive electronic signals from a cable 30 that follows the hose 16 and is coupled to electrodes 38 that sense brain activity in the patient.

In another embodiment of the invention, the electronic device 28 may produce an electrical signal representing sound. In this case, the signal may be supplied to the cable 30 which follows the hose 16 and is connected to a sound transducer 36 attached to the head strap 31. Alternatively, the signal may be supplied to a small loudspeaker (not shown) in the housing 12.

In still another embodiment, the device 28 may serve as a power supply for a vibrating element 40 attached to the head strap 31 of the air mask.

FIG. 2 shows the nose mask 18 and the head strap 31 in detail. The hose 16 supplies air to a nose mask 18 which includes a forehead support 32. A head strap 31 extends from the nose mask 18 and forehead support 32 to the back of the patient's head. This strap 31 supports a sound transducer 36, located near the person's ear, a contact electrode 38 placed in electrical contact with the patient's scalp, and a vibrating element 40 in back of the patient's head. All three elements 36, 38 and 40 are connected electrically by a cable 30 (not shown) that follows the hose 16 and is connected to the electrical device 28 in the housing 12.

There has thus been shown and described a novel sleep inducing apparatus for a CPAP machine which fulfills all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

What is claimed is:

1. A continuous positive airway pressure (“CPAP”) machine having an electrically driven air pump, an air hose coupled to the air pump, and a air mask coupled to the air hose and adapted to be applied to a head of a patient to supply a stream of air to breathing passages of said patient while the patient is sleeping, the improvement comprising a device, attached to said machine, for inducing sleep in said patient.

2. The CPAP machine defined in claim 1, wherein the air mask includes a nose mask and a head strap, attached to said nose mask, for retaining the nose mask on the head of the patient, and wherein at least a part of said sleep inducing device is attached to at least one of said head strap and said nose mask.

3. The CPAP machine defined in claim 1, wherein said air pump is disposed in a housing and wherein a least a part of said sleep inducing device is disposed in said housing.

4. The CPAP machine defined in claim 1, wherein said sleep inducing device comprises a source of a vapor which induces sleep, and a vapor outlet, connected to said source, for supplying said vapor to said air stream.

5. The CPAP machine defined in claim 4, wherein said air pump and said source of vapor are disposed in a common housing.

6. The CPAP machine defined in claim 5, wherein said source of vapor is a supply tank containing at least one of a liquid and a gas.

7. The CPAP machine defined in claim 4, wherein said sleep inducing device further comprises a valve, coupled to said vapor outlet, for controlling said vapor supplied to said air stream.

8. The CPAP machine defined in claim 7, wherein said valve is operative to control a rate at which said vapor is supplied to said air stream.

9. The CPAP machine defined in claim 7, wherein the air mask includes a nose mask and a head strap, attached to said nose mask, for retaining the nose mask on the head of the patient, and further comprising brain electrodes, coupled to at least one of said head strap and said nose mask, for sensing brain activity, and a control device, coupled to said brain electrodes, for controlling said valve.

10. The CPAP machine defined in claim 1, wherein said sleep inducing device comprises a sound generator for producing sleep inducing sounds.

11. The CPAP machine defined in claim 10, wherein said sound generator includes an electronic device for producing an electrical signal representing sound, and at least one transducer, electrically coupled to said electronic device for producing sound in response to said electrical signal.

12. The CPAP machine defined in claim 11, wherein the air mask includes a nose mask and a head strap, attached to said nose mask, for retaining the nose mask on the head of the patient, and wherein said transducer is attached to said head strap.

13. The CPAP machine defined in claim 11, wherein said air pump and said electronic device are disposed in a common housing.

14. The CPAP machine defined in claim 8, wherein said sounds are selected from the group consisting of white noise, ocean beach sounds, train sounds, water sounds, wind sounds, music, spoken words and bird songs.

15. The CPAP machine defined in claim 1, wherein said sleep inducing device comprises a vibrating element for massaging a body part of the patient.

16. The CPAP machine defined in claim 15, wherein the air mask includes a nose mask and a head strap, attached to said nose mask, for retaining the nose mask on the head of the patient, and wherein said vibrating element is attached to said head strap.

17. The CPAP machine defined in claim 15, wherein said vibrating element is electrically actuated.

18. The CPAP machine defined in claim 16, wherein said vibrating element is electrically connected to a source of electrical power and wherein said air pump and said electrical power source are disposed in a common housing.

19. The CPAP machine defined in claim 16, wherein said vibrating element is disposed on said head strap in a position such that it is pressed against a rear portion of the head of the patient when the air mask is applied to the head.

20. The CPAP machine defined in claim 1, wherein said sleep inducing device includes at least one brain electrode attached to said head strap.

21. The CPAP machine defined in claim 20, further comprising an electronic device for generating an electrical signal and a cable for supplying said electrical signal to said electrode.

22. The CPAP machine defined in claim 20, further comprising a cable, coupled to said electrode, and an electronic device, connected to said cable, for processing an electrical brain activity signal derived from said electrode.
